(19) United States
(12) Patent Application Publication
(10) Pub. No.: US 2015/0094591 A1
(43) Pub. Date: Apr. 2, 2015

(54) SPECKLE AND NOISE REDUCTION IN ULTRASOUND IMAGES

(71) Applicant: eagleyemed, Inc., Santa Clara, CA (US)

(72) Inventor: Harish P. HIRIYANNAIAH, San Jose, CA (US)

(21) Appl. No.: 14/563,456

(22) Filed: Dec. 8, 2014

Related U.S. Application Data
(63) Continuation of application No. 14/291,590, filed on May 30, 2014.
(60) Provisional application No. 61/829,891, filed on May 31, 2013.

(51) Int. Cl. A61B 8/08; A61B 8/00; A61B 8/14

(52) U.S. Cl. CPC A61B 8/5269; A61B 8/14; A61B 8/4488; A61B 8/5207
USPC 600/447

(57) ABSTRACT

An ultrasound imaging system includes features to reduce speckle and time gain compression noise. A handheld ultrasound system may include beam forming electronics and digital waveform generators to generate the transmitted pulses with fine grained apodization to improve coherence and reduce speckle. Speckle filtering may be included in the ultrasound system. Features to reduce quantization noise and improve the time gain compression response may be provided.

### CROSS REFERENCE TO RELATED APPLICATIONS

The present application is a Continuation of U.S. application Ser. No. 14/291,590, filed on May 30, 2014, which claims the benefit of U.S. Provisional Application No. 61/829,891, filed on May 31, 2013, the contents of both are hereby incorporated by reference.

### FIELD OF THE INVENTION

The present invention is generally related to techniques to reduce noise and improve image quality in ultrasound medical images.

### BACKGROUND OF THE INVENTION

FIG. 1 illustrates an ultrasound medical image. Noise in ultrasound medical images presents several different aspects. Some types of noise can enhance the visual contrast between tissues. However, the noise also presents other disadvantages, particularly in a telemedicine application.

Although there are different types of noise in ultrasound images, generally speaking the near field may have graininess caused by speckle noise. The far field may have noise associated with time gain compression (TGC) and quantization noise.

Ultrasound images are thus inherently noisy and exhibit two major types of noise, speckle noise, time gain compression (TGC), and quantization noise. Speckle noise is a function of the tissue being imaged and is caused by the reflection of a partially coherent ultrasound wave front travelling through the tissue medium.

TGC and quantization noise is related to compensation of tissue attenuation in the digitized scan lines. In an ultrasound system the transmitted signal is rapidly attenuated in biological tissues and suffers a very large attenuation in a round trip. Tissue attenuation is typically 1 db per MHz per cm. In many commercial systems a set number of TGC adjustments are permitted, such as 6 or 7 TGC adjustment levels over a scan line. As a result the TGC process introduces amplification of noise in a poor signal environment, which is then compounded by quantization noise.

These noise sources can significantly affect the image quality needed for diagnosis and also the compressability of ultrasound streams for network transport. In particular, conventional ultrasound images have a high entropy content. In practical terms, this means that it is difficult to achieve high compression ratios (rates). This, in turn makes it difficult, when network conditions are poor, to send a good quality live video stream of ultrasound images to a remote location.

Conventional ultrasound imaging systems also suffer from other limitations which directly and indirectly influence image quality. FIG. 2 illustrates a conventional ultrasound imaging machine the cable is typically several meters long (e.g., 2 m) and contains 48 to 256 micro-coaxial cables, where the number of micro-coaxial cables scales with the number of transducer elements in the transducer probe. The micro-coaxial cables are expensive and have other disadvantages. In particular, the micro-coaxial cables introduce a cable loss and a cable impedance. For example, a conventional 2 m cable might have a capacitance of 203 pF, while a transducer element could have a capacitance on the order of 5 pF. Additionally, a 2 m cable may introduce a 2 dB attenuation. The cable introduces a large capacitive loading, which makes it impractical to perform fine grained temporal and spatial apodization of the transmitted voltage pulses sent to the transducer probe. This, in turn, reduces the coherence of the ultrasound wavefront, making it difficult to reduce speckle. Additionally, as previously described in the prior art there are typically only 6 or 7 TGC adjustment levels over the scan lines, which introduces quantization errors.

Therefore the present invention was developed in view of the above-described problems.

### SUMMARY OF THE INVENTION

A handheld ultrasound imaging system and method includes features to reduce speckle and time gain compression noise. In one embodiment the handheld ultrasound system includes beam forming electronics and digital waveform generators to generate the transmitted pulses with fine grained apodization to improve coherence and reduce speckle. Speckle filtering may be included in the ultrasound system. Features to reduce quantization noise and improve the time gain compression response may be provided.

One embodiment of a handheld ultrasound imaging system includes a housing, an array of piezoelectric transducers, and beam forming and control electronics to shape a gain and a delay of high voltage pulses coupled to the array of the piezoelectric transducers to drive the array of piezoelectric transducer crystals in a firing sequence with fine grained spatial and temporal apodization to reduce transmitted beam decoherence. Additionally processing electronics is provided for the received ultrasound signal to perform time gain compression (TGC) within the handheld ultrasound system for reflected ultrasound signals received by the array of piezoelectric transducer crystals.

### BRIEF DESCRIPTION OF THE DRAWINGS

FIG. 1 illustrate speckle noise and TGC noise in a conventional ultrasound image.

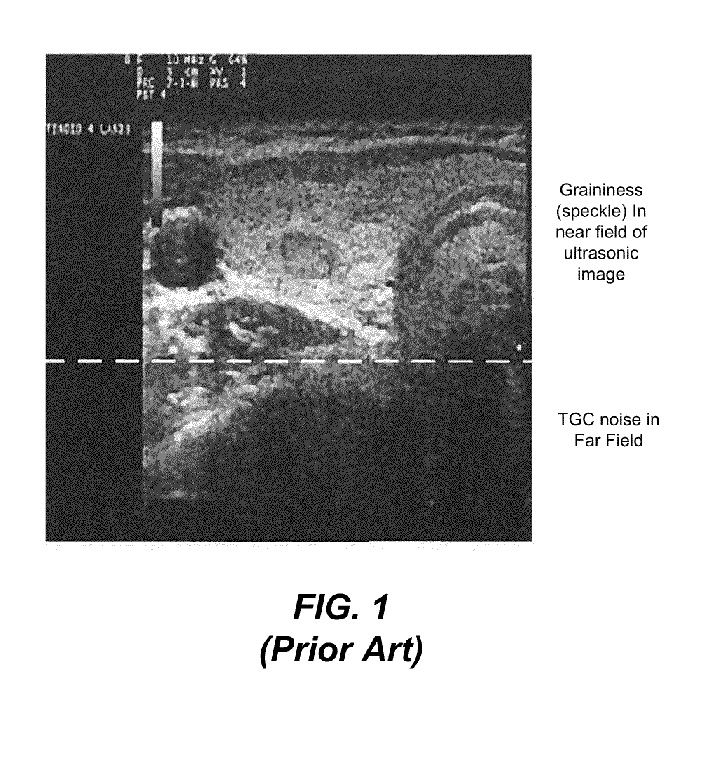

FIG. 2 illustrates a prior art ultrasound imaging system.

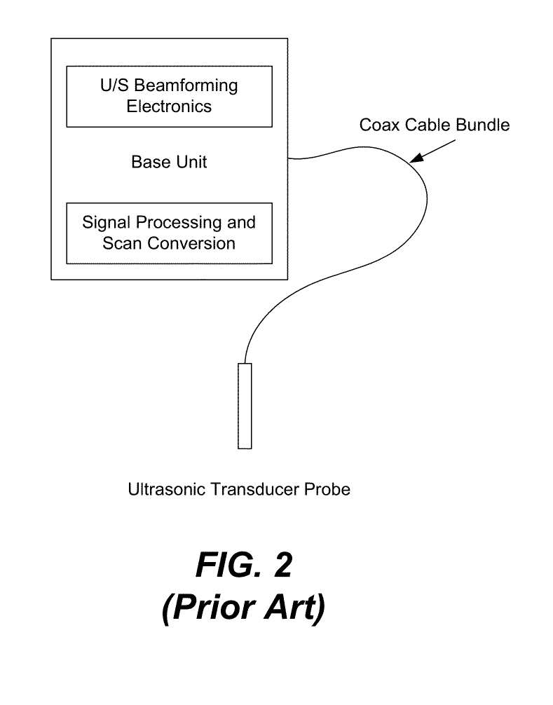

FIG. 3 illustrates a handheld ultrasound system in accordance with an embodiment of the present invention.

FIG. 4 illustrates the use of digital waveform generators to achieve fine grained apodization in accordance with an embodiment of the present invention.

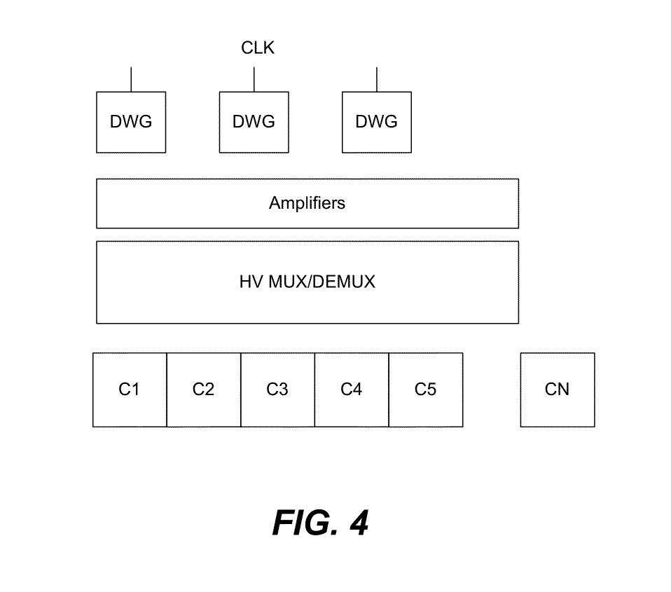

FIG. 5 illustrates speckle noise filtering in accordance with an embodiment of the present invention.

FIG. 6 illustrates aspects of selecting a pixel value for a binned sample in accordance with an embodiment of the present invention.

### DETAILED DESCRIPTION

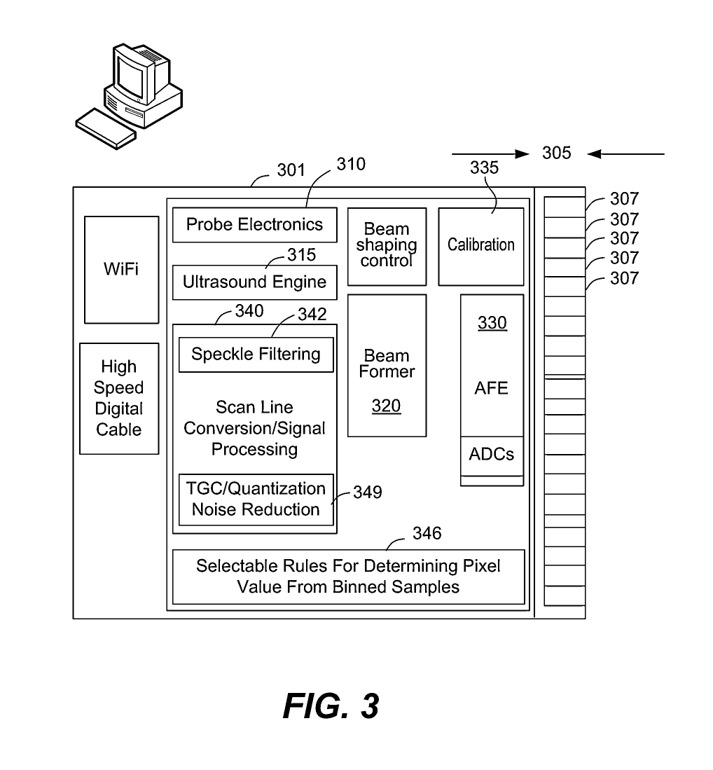

FIG. 3 is a block diagram illustrating aspects of an ultrasound imaging system in accordance with an embodiment of the present invention. The ultrasound imaging system may be used to transmit a live video stream of ultrasound images over a network for real-time review by another doctor. Thus, image quality and compressibility are important considerations.

In one embodiment the ultrasound imaging system is implemented as a hand held ultrasound system including electronics to generate the transmitted ultrasound pulses in a firing sequence and electronics to receive and process the reflected ultrasound pulses. In one embodiment the hand held ultrasound system includes a housing 301, a detachable transducer array 305 having an array of transducer elements 307, such as an array of piezoelectric crystals. The handheld ultrasound system may have a housing 301 that is probe shaped. It will also be understood that the handheld ultrasound system of the present invention may have a housing with a probe shape and size similar to that described in commonly owned U.S. patent application Ser. No. 14/214,370, which is incorporated by reference.

The handheld ultrasound system includes probe electronics 310, an ultrasound engine 315, a beam former 320 and associated beam shaping control electronics 325, an analog front end (AFE) 330 and analog-to-digital converters for the received signal, an auto-calibration section 335, and scan line conversion and signal processing 340. One or more processors are included in the handheld ultrasound system, along with associated memory. The handheld ultrasound system outputs an ultrasound image stream, such as through a wireless (WiFi) or digital cable (e.g. USB). In one embodiment the handheld ultrasound system includes speckle filtering 342, TGC noise reduction 344, and selectable rules for determining pixel values from binned samples 346.

Speckle noise is typically prominent in the near and midfield of an ultrasound image where the TGC gain related artifacts do not overwhelm the signal. Speckle noise in an ultrasound imaging system is associated with diffraction of partially coherent ultrasound waves. Additionally speckle noise is characterized in that it is time varying noise that is non-stationary.

Referring to FIG. 3, in one embodiment the handheld ultrasound system includes electronics to improve the temporal and spatial apodization of the transmitted ultrasound beam to improve coherence and thus reduce speckle. Digital waveform generators (DWGs) generated digital representation of waveforms that are amplified and coupled by a high voltage mux to individual elements of the transducer array in each cycle of a firing sequence. The DWGs are used to provide accurate control of the waveforms provided to each piezoelectric element (C1, C2 . . . CN) fired in a transmit mode of a cycle of the firing sequence. For example, at some time T0, a first set of crystal elements is fired, at time T1, a second set of crystal elements is fired, and so on, with appropriate gaps in time to detect the reflected ultrasound signals. The envelope of the transmitted pulses is represented by a sequence of samples in the pulse envelope coupled to each transducer element. Increasing coherence in the near field reduces speckle.

Coherence can be increased by provide tight apodization in the temporal and spatial domains for that each transducer element that is fired That is, coherence increases when there is precise control of the amplitude and phase of each transducer element that is fired. During a transmit mode, the high voltage (HV) pulse amplitude and phase are scaled by gain and offset corrections and natural focus of the crystals, to increase planarity of the ultrasound wavefront and minimize beam de-coherence. Beam shaping is also accurately controlled by locking the ultrasound frequency with the HV pulser waveform.

In one embodiment the use of clocked DWGs to generate the transmit waveforms aids in achieving precise control. In one embodiment tight control of the amplitude and phase of the HV pulser includes a precision to better than 1 ns time delay, 0.1 degrees in phase, and at least 0.1% in relative gain change.

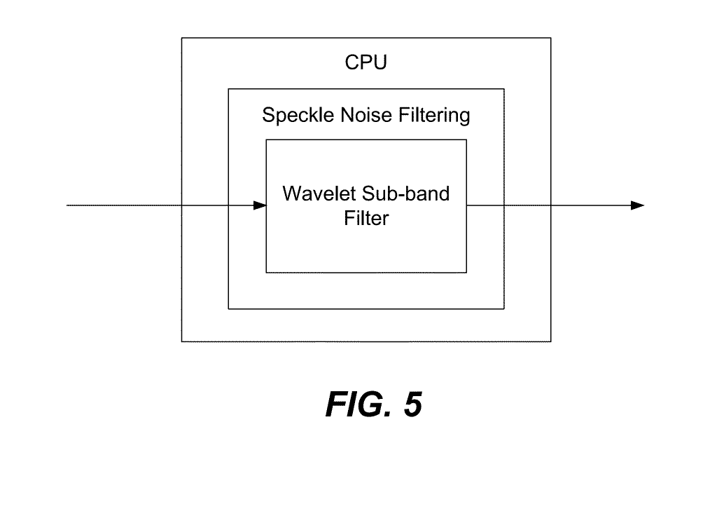

FIG. 5 illustrates speckle noise filtering for the reflected (received) ultrasound signal in accordance with an embodiment of the present invention. Speckle is a time-varying noise that is non-stationary. Speckle noise has high frequency components and is not present in all frequency bands. In one embodiment speckle noise is selectively filtered. In one embodiment a 3 to 4 level wavelet filter is employed in a pyramidal decomposition to segment the frequency bands, either in the 1-D scan-line domain or in the 2-D scan-converted image frame. Based on the nature of the tissue being imaged, a priori, selected frequency bands in the pyramidal decomposition are filtered out. In one embodiment radix 2 wavelet filters are used in the frequency domain The speckle filtering may be performed in a central processing unit of the handheld ultrasound system.

In one embodiment the speckle noise reduction includes sub-frequency filtering that is one-sided wavelet filtering of the scan line. The scan line is then converted into an image.

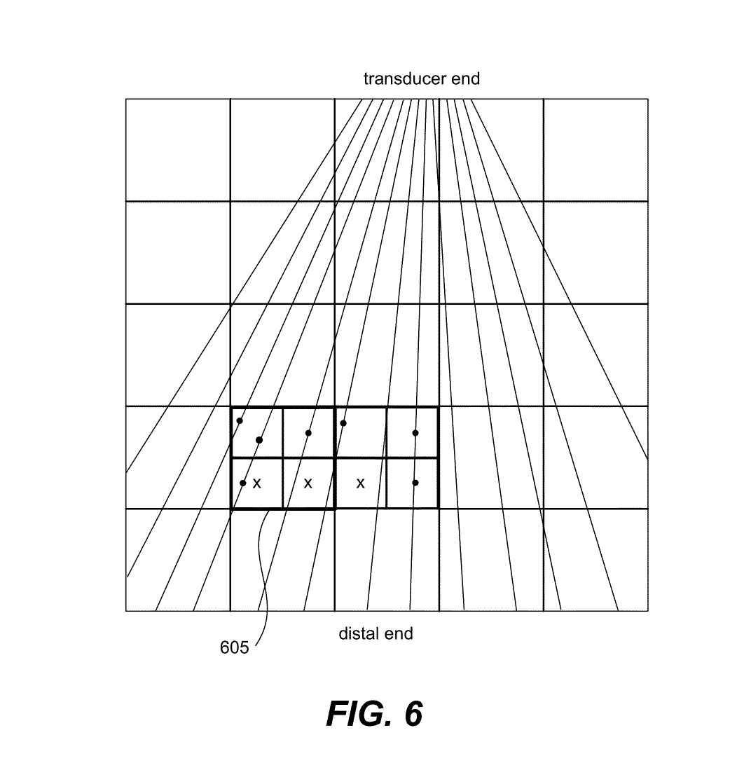

Referring to FIG. 6, in one embodiment in the image grid the scan lines have associated samples at pixel locations, such as a group of pixel bins in region 605. Additionally, there may also be interpolated samples. An individual pixel bin may have more than one sample such that a rule is applied to determine a single pixel value, which may be gray scale value or a color value (for color Doppler ultrasound). Examples of rules include defining the pixel value based on the average, max, min, root mean square, or median of samples that fall in bin. In one embodiment this rule is selectable by a clinician. For example, selecting a “max” would ordinarily generate a more speckled looking image than selecting an “average.” In one embodiment a clinician may select a preference for one of any of the different options. However, more generally a clinician may be provided with only a subset of at least two choices for choosing the binning strategy.

In one embodiment the ultrasound imaging includes one or more features to reduce TGC and quantization noise in the receive mode. In an ultrasound system there is high attenuation of the ultrasound signal within biological tissues. Time gain compression techniques are used to partially compensate for the attenuation. In one embodiment high resolution analog to digital (ADCs) are used during the digitization of the received signals. In one implementation at least 14-bit, and preferably 16-bit ADCs, are employed during the digitization of the signals from the transducer crystals during receive phase. In one embodiment, subsequent beam forming calculations in the digital domain are performed in floating point arithmetic and curve fitting is performed to provide a smooth TGC curve in floating point arithmetic. In one embodiment the smoothed TGC curve is generated by a waveform generator. In one embodiment the subsequent time-varying matched filtered scan-line output is performed in floating point arithmetic. The interpolated scan-line binning and log normalization is maintained in floating point. Additionally, all brightness and contrast changes may be applied to floating point image buffers.

While an exemplary apparatus has been described, additional details on an implementation of a portable ultrasonic probe is described in commonly owned U.S. patent application Ser. No. 14/214,370 “Ultrasound Probe”, filed on Mar. 14, 2014, which is incorporated by reference.

Some additional aspects and benefits of embodiments of the present invention will now be described. Reducing speckle can improve image quality. Additionally, compressibility is a problem in high entropy content ultrasound images. Reducing speckle noise thus improves compressibility by reducing the entropy of the images. Thus, image quality can be improved along with improving compressibility for transport of a live stream of ultrasound images.

While the invention has been described in conjunction with specific embodiments, it will be understood that it is not intended to limit the invention to the described embodiments. On the contrary, it is intended to cover alternatives, modifications, and equivalents as may be included within the spirit and scope of the invention as defined by the appended claims. The present invention may be practiced without some or all of these specific details. In addition, well known features may not have been described in detail to avoid unnecessarily obscuring the invention. In accordance with the present invention, the components, process steps, and/or data structures may be implemented using various types of operating systems, programming languages, computing platforms, computer programs, and/or general purpose machines. In addition, those of ordinary skill in the art will recognize that devices of a less general purpose nature, such as hardwired devices, field programmable gate arrays (FPGAs), application specific integrated circuits (ASICs), or the like, may also be used without departing from the scope and spirit of the inventive concepts disclosed herein. The present invention may also be tangibly embodied as a set of computer instructions stored on a non-transitory computer readable medium, such as a memory device.

What is claimed is:

1. A method of improving image quality in a handheld ultrasound imaging system including piezoelectric transducer having an array of piezoelectric crystals, comprising:
generating transmitted ultrasound pulses including generating high voltage pulses within the handheld ultrasound system in a firing sequence selected to drive the array of piezoelectric transducer crystals with fine grained spatial and temporal apodization selected to reduce transmitted beam decoherence;
receiving and processing reflected ultrasound pulses in the handheld ultrasound imaging system, including performing time gain compression (TGC) within the handheld ultrasound system for reflected ultrasound signals received by the array of piezoelectric transducer crystals; and
outputting an ultrasound image stream from the handheld ultrasound system.

2. The method of claim 1, wherein the reflected ultrasound signals are detected in analog-to-digital converters having at least a 16 bit accuracy.

3. The method of claim 1, wherein performing TGC within the handheld ultrasound system including performing TGC in a smoothed TGC gain curve.

4. The method of claim 3, wherein the smoothed TGC gain curve is represented in a floating point representation.

5. The method of claim 4, further comprising performing brightness and contrast changes in floating point image buffers.

6. The method of claim 4, further comprising performing interpolated scan line binning in floating point arithmetic.

7. The method of claim 4, further comprising performing filtering of scan line output from a beam former in floating point arithmetic.

8. The method of claim 1, wherein generating high voltage pulses includes utilizing digital waveform generators in the handheld ultrasound system to generate digital waveforms for firing the array of piezoelectric crystals in the firing sequence.

9. The method of claim 1, wherein the fine grained apodization includes controlling a phase offset by at least 0.1 degree and at least 0.1% gain over each piezoelectric fired in a firing sequence.

10. The method of claim 1, wherein the fine grained apodization further includes selecting the amplitude and phase of a transmitted pulse to increase planarity of the ultrasound wavefront and minimize de-coherence.

11. The method of claim 1, further comprising performing speckle noise filtering in the handheld ultrasound system.

12. The method of claim 11, wherein the speckle noise filtering includes a multi-level wavelet filter.

13. The method of claim 12, wherein the speckle noise filtering segments frequency bands to selectively filter speckle noise.

14. The method of claim 13, wherein the speckle noise filtering is performed in a scan line domain.

15. The method of claim 14, wherein the speckle noise filtering is performed in a scan converted image frame.

16. The method of claim 1, further comprising selecting a rule for determining a pixel value from samples in a pixel bin based on a clinician preference, wherein the rule is selected from a set of rules including at least two members from the group consisting of a min, a max, an average, a mean, a median, and a root mean square.

17. A handheld ultrasound system, comprising:
a housing;
electronics disposed in the housing to generate transmitted ultrasound pulses, including:
an array of piezoelectric transducers, wherein each piezoelectric transducer includes a piezoelectric crystal; and
beam forming and control electronics to shape a gain and a delay of high voltage pulses coupled to the array of the piezoelectric transducers to drive the array of piezoelectric transducer crystals in a firing sequence chosen for fine grained spatial and temporal apodization to reduce transmitted beam decoherence; and
electronics to receive and process the reflected ultrasound pulses into an ultrasound image stream, including processing electronics for the received ultrasound signal to perform time gain compression (TGC) within the handheld ultrasound system for reflected ultrasound signals received by the array of piezoelectric transducer crystals;
wherein the handheld ultrasound system is configured to output the ultrasound image stream.

18. The handheld ultrasound system of claim 17, further comprising analog-to-digital converters having at least a 16 bit accuracy to detect the reflected ultrasound signal.

19. The handheld ultrasound system of claim 17, wherein the signal processing electronics in the TGC perform TGC in a smoothed TGC gain curve.

20. The handheld ultrasound system of claim 17, wherein the smoothed TGC gain curve is represented in a floating point representation.

21. The handheld ultrasound system of claim 20, wherein the system performs brightness and contrast changes in floating point image buffers.

22. The handheld ultrasound system of claim 20, wherein the processing electronics performs interpolated scan line binning in floating point arithmetic.

23. The handheld ultrasound system of claim 20, wherein the processing electronics filters a scan line output in floating point arithmetic.

24. The handheld ultrasound system of claim 17 wherein the fine grained apodization includes controlling a phase offset by at least 0.1 degree and at least 0.1% gain over each piezoelectric fired in a firing sequence.

25. The handheld ultrasound system of claim 17, wherein the fine grained apodization further includes selecting the amplitude and phase of a transmitted pulse to increase planarity of the ultrasound wavefront and minimize de-coherence.

26. The handheld ultrasound system of claim 17, further comprising a speckle noise filter in the handheld ultrasound system.

27. The handheld ultrasound system of claim 26, wherein the speckle noise filter includes a multi-level wavelet filter.

28. The handheld ultrasound system of claim 26, wherein the speckle noise filter segments frequency bands to selectively filter speckle noise.

29. The handheld ultrasound system of claim 26, wherein the speckle noise filter performs filtering in at least one of a scan line domain and a scan converted image frame.

30. A handheld ultrasound system, comprising:
a housing;
electronics disposed in the housing to generate transmitted ultrasound pulses, including:
an array of piezoelectric transducers, wherein each piezoelectric transducer includes a piezoelectric crystal; and
beam forming and control electronics to shape a gain and a delay of high voltage pulses coupled to the array of the piezoelectric transducers to drive the array of piezoelectric transducer crystals in a firing sequence chosen for fine grained spatial and temporal apodization to reduce transmitted beam decoherence, wherein the fine grained apodization includes controlling a phase offset by at least 0.1 degree and at least 0.1% gain over each piezoelectric transducer fired in the firing sequence; and
digital waveform generators in the handheld ultrasound system to generate digital waveforms for firing the array of piezoelectric crystals in the firing sequence; and
electronics disposed in the housing to receive and process reflected ultrasound pulses into an ultrasound image stream including:
processing electronics for the received ultrasound signal to perform time gain compression (TGC) within the handheld ultrasound system for reflected ultrasound signals received by the array of piezoelectric transducer crystals, wherein the signal processing electronics in the TGC perform TGC in a smoothed TGC gain curve; and
a speckle noise filter to perform speckle noise filtering;
wherein the handheld ultrasound system is configured to output the ultrasound image stream.
